(19) United States
(12) Patent Application Publication
(10) Pub. No.: US 2008/0031979 A1
(43) Pub. Date: Feb. 7, 2008

(54) USE OF EXTRACTS FOR THE TREATMENT OF VIRAL DISORDERS

(76) Inventors: Claude Saliou, Bernardsville, NJ (US); Sekhar Boddupalli, Palo Alto, CA (US); Khalid Mahmood, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/462,384

(22) Filed: Aug. 4, 2006

(51) Int. Cl. A61K 36/82; A61K 36/185

(52) U.S. Cl. 424/729; 424/769

(57) ABSTRACT

The present invention relates to plant extracts and compositions containing such plant extracts useful in the treatment of viral disorders, including but not limited to the treatment of viral lesions resulting from viruses such as Herpes Simplex virus.

### BACKGROUND INFORMATION

The present invention relates to the use of a combination of extracts from pomegranate and green tea for the treatment of viral disorders. In one embodiment, the invention relates to the treatment of viral lesions, for example, the treatment of cold sores resulting from the herpes simplex virus (HSV), with compositions containing such plant extracts.

Cold sores are often associated with an unpleasant stigmatism; however, the great number of individuals affected by the virus confirms it as a relevant virus in our society. Also referred to as fever blisters, cold sores are brought on by the herpes simplex virus which resides in the nerves of the cold sore sufferers. The herpes simplex virus is part of the herpes virus group and shares the distinct characteristic of the ability to lie dormant within the body, specifically in the nerve cells, for long periods of time, or for the lifetime of the individual.

Cold sores are contagious and reoccurring. The first outbreak often occurs 1 to 3 weeks after the virus has initially been contracted. The virus most often spreads through contact with the open sores of an infected individual. However, the virus can also spread even in the absence of open sores or any symptoms. The sores initially appear as small, fluid-filled blisters on the skin.

Symptoms include, but are not limited to, fever, muscle aches, swollen glands, malaise, itching, inflammation, irritation, pain, swelling and burning. These symptoms are followed by an initial tingling sensation to the sufferer then followed by painful blisters. The usual duration of the sores can last from 2 to 3 weeks with the blisters scabbing and then eventually falling off the skin completely, generally without any scarring of the infected skin area.

Causes for the outbreaks include a weakening of the immune system from colds or other infections, the length of time the person infected has had the virus, exhaustion, emotional and physical stress, the menstrual cycle, immunosuppression, overexposure to wind and sunlight, and drug and heavy alcohol use.

The most common types of this virus are herpes simplex virus-1 (HSV-1) and herpes simplex virus-2 (HSV-2). The main difference between the two viruses is where they set up their dormancy site in the body. HSV-1 usually lies dormant in the trigeminal ganglion, the nerve cells located around the ear, whereas HSV-2 usually lies dormant in the sacral ganglion, the nerve cells located at the base of the spine.

HSV-1 is responsible for the formation of cold sores formed around the lips and less infrequently, the chin, nostrils, fingers and the gums and roof of the mouth of an infected individual. Extremely uncommon, although possible and less known to the public, is the formation of cold sores in the genital area from HSV-1 sufferers. Dismissed by the public as more socially acceptable and generally more of an inconvenience than a health risk, HSV-1 can cause serious dangers to an infected individual. HSV-1 can spread to the eye causing ocular herpes, which can cause blindness. HSV-1 also has the ability to spread to the brain, causing herpes encephalitis, which can lead to death.

The most infamous of the herpes simplex viruses, HSV-2 is responsible for cold sores in the genital area. Approximately 1 in 4 individuals are believed to be infected with HSV-2. HSV-2 can also be spread to the eye and brain, as discussed above. Not only is HSV-2 an uncomfortable and often painful physical affliction, it can cause emotional and psychological suffering to the affected individual.

Herpes simplex virus-3 (HSV-3), also known as the varicella-zoster virus, causes chickenpox and later, shingles. Varicella is the primary infection that causes chickenpox. Chickenpox initially appears as small, red bumps on the abdomen, chest or face, later forming into blisters that eventually scab and fall off the body. Although symptoms do not show until about two days after exposure to the virus, the symptoms can last from five to ten days and include a red itchy rash, fever and headache.

Highly contagious, chickenpox is spread by an infected individual, most often through sneezing, coughing and breathing. It is then inhaled in the newly infected individual's lungs, passing into the bloodstream. Entering the nerve cells, the virus lays dormant for years, even a lifetime, possibly reappearing as herpes zoster or shingles. Shingles is thought to be only contracted from an individual with chickenpox, never from someone with shingles. The initial symptoms of shingles usually include a tingling feeling, itchiness, numbness, or stabbing pain in or under the skin. Shingles generally affects one side of the body, characterized by an outbreak of severely painful and itchy blisters. Postherpetic neuralgia can occur as a painful after effect of shingles. Treatments for postherpetic neuralgia include steroids, antiviral drugs, antidepressants, anticonvulsants, and topical agents.

Antivirals such as acyclovir, valcyclovir or farcyclovir can be used to treat oral and genital HSV-1 and HSV-2, as well as shingles and chickenpox resulting from HSV-3. These antivirals reduce the amount of time that it takes for the blisters to heal and can be taken orally on a regular basis in order to prevent reoccurrences. However, these existing drugs may cause side effects such as, for example, nausea, vomiting, diarrhea, headache, dizziness, and/or rashes, and some users may experience disorientation, hallucinations, delirium and tremors. Abnormal renal function can also occur as a result of acyclovir, valcyclovir or farmcyclovir administration. Patients with preexisting renal dysfunction or dehydration, or with hepatic dysfunction are advised to use it with caution. Abnormal renal function can also occur due to drug interactions with nephrotoxic drugs, some pain medicines, and cyclosporine.

Therefore, new effective and safe compositions to treat viral lesions, such as cold sores, by minimizing and/or eliminating the number and/or severity of lesions, are desirable.

### SUMMARY OF THE INVENTION

The present invention relates to compositions including an extract of pomegranate and an extract of green tea and methods for using such plant extracts, such as in the treatment of viral lesions.

In one embodiment, the present invention relates to a method for the treatment of viral lesions including administering to a subject in need of such treatment a composition containing a composition including an extract of pomegranate and an extract of green tea.

In some embodiments, the viral lesions result from a virus selected from a herpes virus (e.g., HSV-1, HSV-2, or HSV-3). In other embodiments the viral lesions are cold sores.

In some embodiments, the method of administration is topical, such as topically applying the extract/composition to the lesion. In other embodiments the method of administration is oral.

In another aspect, the invention relates to a method for the treatment of one or more symptoms associated with viral infections in a subject suffering from a virus, such as a herpes simplex virus, with a composition including an extract of pomegranate and an extract of green tea. In some embodiments, the symptoms include fever, muscle aches, swollen glands, malaise, itching, inflammation, irritation, pain, swelling and burning.

In another embodiment, the invention relates to a method for controlling viral growth and replication resulting from herpes simplex virus, including administering to the subject in need of such treatment, a composition including an extract of pomegranate and an extract of green tea.

In other embodiments, the invention relates to the method of treatment with a composition that further contains at least one of the following: (i) a skin protectant active ingredient selected from allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, and/or zinc oxide; (ii) an external, anesthetic or antipruritic ingredient selected from benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate and/or diphenydramine hydrochloride; and/or (iii) an other ingredient selected from allyl isothiocyanate, ammonia solution, aspirin, bismuth sodium tartrate, capsaicin, capsicum oleoresin, chloral hydrate, chlorobutanol, cyclomethycaine sulfate, eucalyptus, eugenol, glycol salicylate, hexylresorcinol, histamine dihydrochloride, metapyriline hydrochloride, methyl nicotinate, methyl salicylate, pectin, salicylamide, tannic acid, thymol, trolamine salicylate, turpentine oil, zinc sulfate, aluminum acetate, aluminum sulfate, sucrose stereate, sucrose distereate, and/or witch hazel.

In other embodiments, the invention relates to the method of treatment with a composition that further contains one or more agents selected from the group consisting of anti-microbial agents, other antiviral agents, antifungal agents, antioxidants, anti-inflammatory agents, soothing agents, buffering agents, sunscreens, cosmetic agents, fragrances, lubricants, moisturizers, drying agents, and thickening agents.

### DETAILED DESCRIPTION OF THE INVENTION

### Definitions

As used in the present specification, the following words and phrases are generally intended to have the meanings as set forth below, except to the extent that the context in which they are used indicates otherwise.

The term “astringent active ingredients” includes but is not limited to, aluminum acetate, aluminum sulfate, and witch hazel.

The term “cosmetics” includes make-up, foundation, and skin care products. The term “make-up” refers to products that leave color on the face, including foundations, i.e., concealers, lip balms, lipsticks and so forth. The term “foundation” refers to liquid, creme, mousse, compact, concealer, or like products that even out the overall coloring of the skin. Foundation is typically manufactured to work better over moisturized and/or oiled skin. The term “skin care products” refers to products used to treat or otherwise care for, moisturize, improve, or clean the skin. The term “cosmetics” may also include other safe skin protectant drug products for over-the-counter human use as defined in the code of federal regulations such as 21 CFR 347 and 21 CFR 348.

The term “effective amount” refers to that amount of a extract/compound/composition of the present invention that is sufficient to effect treatment, as defined herein, when administered to a mammal in need of such treatment. The effective amount will vary depending upon the subject and disease condition being treated, the weight and age of the subject, the severity of the disease condition, the particular extract chosen, the dosing regimen to be followed, and the like, all of which can readily be determined by one of ordinary skill in the art.

The term “external analgesic, anesthetic, and antipruritic active ingredients” includes, but is not limited to, benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate and diphenydramine hydrochloride.

The term “inflammation” refers to the localized protective response elicited by the destruction of tissues. It is characterized by signs of pain, heat, redness, and/or swelling.

The term “pharmaceutically acceptable” refers to those extracts, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

The term “skin care products” may include, but is not limited to, skin protectant active ingredients, astringent active ingredients, external analgesic, anesthetic and antipruritic active ingredients as published in 21 CFR 347.10, 347.12 and 348.10, and other ingredients as published in 55 FR 3370, or mixtures thererof.

The term “skin protectant active ingredients” include but are not limited to allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, and zinc oxide. Skin protectant active ingredients may also include sunscreen agents.

The term “sunscreen” may include, but is not limited to, organic or inorganic sunscreens, sun blocks titanium oxide and zinc oxide, and skin protectants and/or mixtures thereof. Sunscreen products providing a minimum SPF value of not less than 2, include, but are not limited to, aminobenzoic acid (PABA); avobenzone, cinoxate, dioxybenzone, homosalate, menthyl anthranilate, methoxycinnamate, octocrylene, octyl methoxycinnamate, octyl salicylate, oxybenzone, padimate O, phenylbenzimidazole sulfonic acid, sulisobenzone, titanium dioxide, trolamine salicylate, titanium oxide, and zinc oxide.

The term “topical application” means directly laying on or spreading on outer skin using, e.g., by use of the hands or an applicator such as a wipe, puff, roller, or spray. As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

The term “treatment” or “treating” means any treatment of a disease or disorder in a mammal, including: (i) inhibiting the disease or disorder, that is, arresting or suppressing the development of clinical symptoms of the disease or disorder; and/or (ii) relieving the disease or disorder, that is, causing the regression or cure of clinical symptoms of the disease or disorder, and/or (iii) accelerating the healing of the lesions, and/or (iv) preventing or protecting against the disease or disorder, that is, causing the clinical symptoms not to develop. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” or “prophylactic” is intended as an element of “treatment” to encompass both “preventing” and “suppressing” as defined herein. The term “protection,” as used herein, is meant to include “prophylaxis” and “”prevention”. Examples include, but are not limited to, supressing the reoccurence or severity of symptoms of viral infections, such as lesions.

The term “viral lesions” refers to all lesions which have been affected by viral disorders such as all herpes, including, but not restricted to, cold sores, genital herpes, shingles, chicken pox, forms of zoster, and other disorders of viral nature. This term is not restricted to orofacial lesions and includes manifestations on all parts of the body.

### EXTRACTS OF THE INVENTION

The compositions of present invention includes an extract from pomegranate and an extract from green tea. What is meant by a “extract” is a blend of compounds isolated from the plant (e.g., the green tea or pomegranate plant). Such compounds may be isolated from one or more part of the plant (e.g., the whole plant, flower, seed, root, rhizome, stem, fruit and/or leaf of the plant) by physically removing a piece of such plant, such as grinding a flower of the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). Examples of such compounds, include, but are not limited to, Ellagic acid, tannins, caffeine, and catechins such as Epigallocatechin Gallate.

The compositions of the present invention contains an extract of green tea. What is meant by an “extract of green tea” is an extract from a plant of the genus Camellia. Examples of Camellia species include, but are not limited to, sinensis, taliensis, irrawadiensis and japonica. Extraacts of Camellia are described in Chemistry and Applications of Green Tea by Takehiko Yamamoto, Lekh Raj Juneja, D. C. Chu, Mujo Kim, L. R. Juneja, M. Kim (CRC-Press 1997).

The composition of the present invention also contain an extract of pomegranate. What is meant by an “extract of pomegranate” is an extract from a plant of the genus Punica. Examples of Punica species include, but are not limited to, granatum. Extracts of Pomegranate are described in Medicinal Plants of the World, Volume 1 by Ross, Ivan A. (Humana Press 2003).

In one embodiment, the plant extract is present in the composition in an amount from about 0.001% to about 20% by weight, in particular in an amount from about 0.1% to about 10% by weight of the composition. Unless stated otherwise, the weight of the extract refers to the dry weight of the extract.

### Utility, Testing and Administration

### General Utility

Extracts, compositions/formulations and methods of the present invention are useful in treating or managing viral diseases, and/or symptoms thereof. Viral diseases can include, but are not limited to: molluscum contagiosum; human T-cell lymphotropic virus (HTLV); human immuno-deficiency virus (HIV); acquired immuno-deficiency virus (AIDS); human papillomavirus; herpesvirus; herpes; viral dysentery; arenavirus; coronavirus; enterovirus; common cold; flu; measles; rubella; chicken pox; mumps; polio; rabies; mononucleosis; ebola; respiratory syncytial virus; dengue fever; yellow fever; lassa fever; bunyavirus; filovirus; flavivirus; hantavirus; rotavirus; West Nile fever; arbovirus; parainfluenza; smallpox; Epstein-Barr virus; cytomegalovirus; viral gastroenteritis; acute appendicitis; hepatitis (A-E; X); cold sores; meningitis; encephalitis; shingles; pneumonia; Rift Valley fever; hendra fever; roseola; sandfly fever; severe acute respiratory syndrome (SARS); warts; cat scratch disease; slap-cheek syndrome; orf; hand, foot and mouth disease; and pityriasis rosea.

It is an objective of this invention to control viral diseases such as herpes simplex viruses (HSV-1, HSV-2, or HSV-3). It is another objective of this invention to improve the symptoms associated with viral infections, such as but not limited to, fever, muscle aches, swollen glands, malaise, itching, inflammation, irritation, pain, swelling and burning.

It is an objective of this invention to provide improved compositions and methods for the treatment or management of HSV-1 and HSV-2 during the active phase of the virus. It is another objective of this invention to provide compositions that would help clear up and/or reduce the number of cold sores resulting from HSV-1 and HSV-2. It is another objective of this invention to provide compositions to help prevent the outbreak of new cold sores resulting from HSV-1 and HSV-2. It is another objective of this invention to reduce the healing time of the cold sores.

It is another objective of this invention to control viral growth and/or replication resulting from HSV-1 and HSV-2.

It is another objective of this invention to provide compositions and ingredients for compositions that can be used in combination with conventional viral lesion medications to reduce their appearance. It is also an objective of the invention to provide methods for using compositions of the invention with conventional viral lesion treatments for new combination therapies that maximize viral lesion management. It is a corresponding objective to alleviate the negative social and psychological impacts frequently suffered by persons afflicted with HSV-1 and HSV-2.

It is an objective of this invention to provide improved compositions and methods for the treatment and management of HSV-3 during the active phase of the virus. It is another objective of this invention to provide compositions that would help clear up and/or reduce the number of chickenpox resulting from HSV-3.

It is another objective of this invention to provide compositions and ingredients for compositions that can be used in combination with conventional chickenpox medications to reduce their appearance and/or reduce associated inflammation and irritation, including itching. It is also another objective of the present invention to provide methods for using compositions of the invention with conventional chickenpox treatments to provide new combination therapies that maximize chickenpox maintenance.

It is another objective of this invention to provide compositions that would help clear up and/or reduce the number of shingles resulting from HSV-3. It is another objective of this invention to provide compositions and ingredients for compositions that can be used in combination with conventional shingles medications to reduce the appearance of and/or reduce associated symptoms ranging from mild itching to severe and intense pain. It is another objective of the invention to provide methods for using compositions of the invention with conventional shingles treatments to provide new combination therapies that maximize shingles maintenance.

### Testing

This section describes how compositions incorporating extracts of the present invention are selected.

In vitro evaluation of anti-viral activity can be determined by plaque reduction as reported in J. Nat. Prod. (1990) 53, 340-344; or as described in Example 1. To pre-grown Vero cells (ATCC CCL-81 is added a virus suspension (ATCC VR-260) mixed with complete medium containing various concentrations of the test extract and the mixture is incubated until maximum cytopathic effect (CPE) is observed in the untreated virus control culture. The CPE inhibition is determined by adding a dye (MTS, (3-[4,5-dimethylthiazol-2-yl-5]-[3-carboxymethyoxyphenyl]-2-[4-sulfophenyl]-2H tetrazolium)) uptake procedure (Promega's Cell Titer Aqueous One Solution). This method measures cell viability and is based on the reduction of the tetrazolium-based MTS by mitochondrial enzymes of viable host cells to MTS formazan. The purple color of the MTS formazan is then measured spectrophotometrically. The optical density (OD) value of each culture is a function of the amount of formazan produced which is proportional to the number of viable cells. Extracts of the present invention showed superior anti-viral activity as described in Table I.

In vivo evaluation of anti-inflammatory activity can be determined by well characterized assays measuring Carrageenan-Induced Paw Edema and by Mouse Ear Inflammatory Response to Topical TPA (Gabor, M., Mouse Ear Inflammation Models and their Pharmacological Applications, 2000). Carrageenan-Induced Paw Edema is a model of inflammation, which causes time-dependent edema formation following carrageenan administration into the intraplantar surface of a rat paw. The application of 12-O-tetradecanoylphorbol-13-acetate (TPA) to the ears of mice produces immediate vasodilation and erythema, followed by the abrupt development of edema, which is maximal at 5-6 hours. The onset of edema coincides with the extravasations of protein and leukocytes. This assay measures a test extract's ability to treat these inflammatory processes via systemic or topical route of administration.

### Administration

In one embodiment, the extracts of the invention are administered at a pharmaceutically effective amount, e.g., a dosage sufficient to provide treatment for the disease states previously described. Administration of the extracts of the invention can be via any of the accepted modes of administration for agents that serve similar utilities.

In employing the extracts of this invention for treatment of the above conditions, any pharmaceutically acceptable mode of administration can be used. The extracts of the invention can be administered either alone or in combination with other pharmaceutically acceptable excipients, including solid, semi-solid, liquid, or aerosol dosage forms, such as, for example, tablets, capsules, powders, granules, cachets, liquids, suspensions, solutions, suppositories, aerosols, or the like. The extracts of the present invention can also be administered in sustained or controlled release dosage forms, including depot injections, osmotic pumps, pills, transdermal (including electrotransport) patches, and the like, for the prolonged administration of the extract at a predetermined rate, i.e., in unit dosage forms suitable for single administration of precise dosages. The compositions will typically include a conventional pharmaceutical carrier or excipient and a extract of the present invention. In addition, these compositions may include other medicinal agents, pharmaceutical agents, carriers, adjuvants, and the like, including, but not limited to, permeability enhancers and slow release formulations.

Compositions and methods of the invention may be employed in skin care applications where treatment or amelioration of viral lesions is desirable. For example, extracts and compositions of the invention may be incorporated into leave-on preparations: wipes; towelettes; swabs; lotions; salves; gels; creams; oils; ointments; pastes; balms; tinctures; emulsions; colloidal suspensions; lipsticks; and stick compositions.

Compositions useful for topical administration of the compositions of the present invention formulated as solutions typically include a pharmaceutically-acceptable aqueous or organic solvent. The term pharmaceutically-acceptable organic solvent refers to a solvent which is capable of having a composition of the present invention dispersed or dissolved therein, and of possessing acceptable safety properties (e.g., irritation and sensitization characteristics). Examples of suitable organic solvents include: propylene glycol; polyethylene glycol (200-600); polypropylene glycol (425-2025); glycerol; 1,2,4-butanetriol; sorbitol esters; 1,2,6-hexanetriol; ethanol; isopropanol; butanetriol; sorbitol esters; 1,2,6-hexanetriol; ethanol; isopropanol; butanediol; and mixtures thereof.

Topical formulations of the present invention typically contain the novel composition of the invention and optionally, a polar solvent. Solvents suitable for use in the formulations of the present invention include any polar solvent capable of dissolving the novel composition of the invention. Suitable polar solvents include: water; alcohols (such as ethanol, propyl alcohol, isopropyl alcohol, hexanol, and benzyl alcohol); polyols (such as propylene glycol, polypropylene glycol, butylene glycol, hexylene glycol, sorbitol, and glycerin); and panthenol dissolved in glycerin, flavor oils and mixtures thereof. Mixtures of these solvents can also be used. Exemplary polar solvents are polyhydric alcohols and water, such as but not limited to, glycerin, panthenol in glycerin, glycols such as propylene glycol and butylene glycol, polyethylene glycols, water and mixtures thereof.

An emollient may also be added to the topical compositions of the present invention. The emollient component can include fats, oils, fatty alcohols, fatty acids and esters which aid application and adhesion, yield gloss, and most importantly, provide occlusive moisturization. Suitable emollients for use are isostearic acid derivatives, isopropyl palmitate, lanolin oil, diisopropyl dimerate, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristal myristate, triisocetyl citrate, cetyl alcohol, octyl dodecanol, oleyl alcohol, panthenol, lanolin alcohol, linoleic acid, linolenic acid, sucrose esters of fatty acids, octyl hydroxystearate and mixtures thereof. Examples of other suitable emollients can be found in the Cosmetic Bench Reference, pp. 1.19-1.22 (1996), incorporated herein by reference. Suitable emollients include polar emollient emulsifiers (such as linear or branched chained polyglycerol esters) and non-polar emollients.

By “polar emollient,” as used herein, is meant any emollient emulsifier having at least one polar moiety and wherein the solubility (at 30° C.) of the compound in the polar emollient is greater than about 1.5%, greater than about 2%, or greater than about 3%. Suitable polar emollients include, but are not limited to, polyol ester and polyol ethers such as linear or branched chained polyglycerol esters and polyglycerol ethers. Nonlimiting examples of such emollients include polyglyceryl-3-diisosterate, polyglyceryl-2-sesquiisostearate, polyglyceryl-5-distearate, polyglyceryl-10-distearate, polyglyceryl-10-diisostearate, acetylated monoglycerides, glycerol esters, glycerol tricaprylate/caprate, glyceryl ricinoleate, glyceryl isostearate, glyceryl myristate, glyceryl linoleate, polyalkylene glycols such as PEG 600, monoglycerides, 2-monolaurin, sorbitan esters and mixtures thereof.

By “non-polar emollient,” as used herein, means any emollient emulsifier possessing no permanent electric moments. Suitable non-polar emollients include, but are not limited to, esters and linear or branched chained hydrocarbons. Non-limiting examples of such emollients include, but are not limited to, isononyl isononanoate, isopropyl isostearate, octyl hydroxystearate, diisopropyl dimerate, lanolin oil, octyl palmitate, isopropyl palmitate, pariffins, isoparafins, acetylated lanolin, sucrose fatty acid esters, isopropyl myristate, isopropyl stearate, mineral oil, silicone oils, dimethicone, allantoin, isohexadecane, isododecane, petrolatum, and mixtures thereof. The solubility of the compound in polar or non-polar emollients is determined according to methods known in the art.

Oils that act as emollients also impart viscosity, tackiness, and drag properties to cosmetic compositions such as lipstick. Examples of suitable oils include, but are not limited to, caprylic triglycerides; capric triglyceride; isostearic triglyceride; adipic triglyceride; propylene glycol myristyl acetate; lanolin; lanolin oil; polybutene; isopropyl palmitate; isopropyl myristate; isopropyl isostearate; diethyl sebacate; diisopropyl adipate; tocopheryl acetate; tocopheryl linoleate; hexadecyl stearate; ethyl lactate; cetyl oleate; cetyl ricinoleate; oleyl alcohol; hexadecyl alcohol; octyl hyroxystearate; octyl dodecanol; wheat germ oil; hydrogenated vegetable oils; castor oil; petrolatum; modified lanolins; branched-chain hydrocarbons; alcohols and esters; corn oil; cottonseed oil; olive oil; palm kernel oil; rapeseed oil; safflower oil; jojoba oil; evening primrose oil; avocado oil; mineral oil; shea butter; octylpalmitate; maleated soybean oil; glycerol trioctanoate; diisopropyl dimerate; and volatile and non-volatile silicone oils including phenyl trimethicone.

Suitable oils for use herein are acetylglycerides, octanoates, and decanoates of alcohols and polyalcohols, such as those of glycol and glycerol, the ricinoleates of alcohols and polyalcohols such as cetyl ricinoleate, polyglyceryl-3 diisostearate, polyglycerol ethers, polyglyerol esters, caprylic triglycerides, capric triglycerides, isostearic triglyceride, adipic triglyceride, phenyl trimethicone, lanolin oil, polybutene, isopropyl palmitate, isopropyl isostearate, cetyl ricinoleate, octyl dodecanol, oleyl alcohol, hydrogenated vegetable oils, castor oil, modified lanolins, octyl palmitate, lanolin oil, maleated soybean oil, cetyl ricinoleate, glyceryl trioctanoate, diisopropyl dimerate, synthetic lanolin derivatives and branched chain alcohols, sucrose esters of fatty acids, octyl hydroxystearate, and mixtures thereof.

A surfactant may also be added to compositions of the invention, in order to confer beneficial application properties. Surfactants suitable for use are those which can form emulsions and/or association structures. Surfactants suitable for use do not present dermatological or toxicological problems. Anionic surfactants, nonionic surfactants, cationic surfactants, amphoteric surfactants and mixtures thereof are suitable for use. For example, anionic surfactants, nonionic surfactants, cationic surfactants, amphoteric surfactants and mixtures thereof having a Krafft point at or below about ambient temperature are used.

The compositions of this invention may contain one or more materials, herein singly or collectively referred to as a “solidifying agent”, that is effective to solidify the particular liquid base materials to be used in a cosmetic composition. As used herein, the term “solidify” refers to the physical and/or chemical alteration of the liquid base material so as to form a solid or semi-solid at ambient conditions, i.e., to form a final composition that has a stable physical structure and can be deposited on the skin under normal use conditions. As is appreciated by those skilled in the art, the selection of the particular solidifying agent for use in the cosmetic compositions will depend upon the particular type of composition desired, i.e., gel or wax-based, the desired rheology, the liquid base material used and the other materials to be used in the composition.

Liposomal formulations may also be useful for the compositions of the present invention. Such compositions can be prepared by combining a composition of the present invention with a phospholipid, such as dipalmitoylphosphatidyl choline, cholesterol and water according to known methods, for example, as described in Mezei et al., J. Pharm. Pharmacol. 34:473-474 (1982), or a modification thereof. Lipids suitable for forming liposomes may be substituted for the phospholipid, as may be lecithin, as well. The liposome preparation is then incorporated into one of the above topical formulations (for example, a gel or an oil-in-water emulsion) in order to produce the liposomal formulation. Other compositions and pharmaceutical uses of topically applied liposomes are described, for example, in Mezei, M. Topics in Pharmaceutical Sciences, Breimer et al. eds., Elsevier Science, New York, N.Y., pp. 345-358 (1985).

Topical compositions of the present invention may also be applied to the oral cavity when incorporated in mouth rinses or mouthwashes, or may be used for ophthalmic treatment incorporated in eyewashes, eyedrops, or eye swabs.

Another manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions take the form of solutions, suspensions, tablets, dispersible tablets, pills, capsules, powders, sustained release formulations and the like.

Compositions may take the form of a pill or tablet, and thus the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose and derivatives thereof, and the like.

In preparing a formulation, it may be necessary to mill the extracts to provide the appropriate particle size prior to combining with the other ingredients. If the active extract is substantially insoluble, it ordinarily is milled to a particle size of less than 200 mesh. If the active extract is substantially water soluble, the particle size is normally adjusted by milling to provide a substantially uniform distribution in the formulation, e.g., about 40 mesh.

Some examples of suitable excipients for oral preparations include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of the extracts of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can include an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid pharmaceutically administrable compositions can, for example, be prepared by dissolving, dispersing, etc. an active extract as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents; emulsifying agents; solubilizing agents; pH buffering agents and the like, for example, sodium acetate, sodium citrate, cyclodextrine derivatives, sorbitan monolaurate, triethanolamine acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition (1975). The composition or formulation to be administered will, in any event, contain a quantity of the active extract in an amount effective to alleviate the symptoms of the subject being treated.

The liquid forms in which the novel compositions of the present invention may be incorporated for administration orally include aqueous solutions suitably flavored syrups; aqueous or oil suspensions; and flavored emulsions with edible oils such as cottonseed oil, sesame oil, coconut oil, or peanut oil, as well as elixirs and/or similar pharmaceutical vehicles.

Alternatively, liquid or semi-solid oral formulations may be prepared by dissolving or dispersing the active extract in vegetable oils, glycols, triglycerides, propylene glycol esters (e.g. propylene carbonate) and the like, and encapsulating these solutions or suspensions in hard or soft gelatin capsule shells.

This invention includes compositions associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the oral compositions discussed above can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active extract, soft and hard gelatin capsules, suppositories, sterile injectable solutions, and sterile packaged powders.

Parenteral administration can employ the implantation of a slow-release or sustained-release system, such that a constant level of dosage is maintained. The percentage of active extract contained in such parenteral compositions is highly dependent on the specific nature thereof, as well as the activity of the extract and the needs of the subject.

Compositions of the present invention can be used alone or in combination with one or more additional beneficial agent, for example with an anesthetic, an analgesic, an antiinfective, an antibacterial, or an antifungal agent, or mixtures thereof. Some examples of suitable anesthetics that may be added to the compositions of the present invention in order to provide alleviation of pain and itching include, but are not limited to, benzocaine, lidocaine, tetracaine, dyclonine, pramoxine, butamben, camphor, menthol, eucalyptol, thymol, dibucaine, bupivocaine, carbocaine, ropivocaine, procaine, cocaine, novocaine, xylocaine, mepivacaine, benzethonium chloride, anethol, hexetidine, eugenol, caffeine, nicotine, combination of lidocaine and prilocaine, oil of cloves, tea tree oil, lidocaine hydrochloride, dibucaine hydrochloride, tetracaine hydrochloride, tronothane, dyclonine hydrochloride, pramoxine hydrochloride, diperodon, butamben picrate, cyclomethycaine sulfate, cyclomethycaine hydrochloride, dimethisoquin hydrochloride, opoid analgesics such as morphine and its derivatives, and psychoactive drugs including tricyclic antidepressant drugs (TCAs).

Some examples of suitable analgesics that may be added to the compositions of the present invention in order to provide relief from fever, aches and pains that may be associated with the virus, include, but are not limited to, acetaminophen, ibuprofen, aspirin, salicyclamide, trolamine salicylate, methyl salicylate, salicylate salts, N,N-dimethyl aspartic acid, N,N-dimethyl glutamic acid, tripelennamine hydrochloride, hydrocortisone, hydrocortisone acetate and antipyrine.

Some examples of suitable antiinfectives or antibacterials that may also be added to the compositions of the present invention in order to inhibit the spread of infection that may be associated with the virus, include benzalkonium bromide, benzalkonium chloride, chlorhexidine hydrochloride, triclosan, sorbic acid, benzethonium chloride, methyl benzethonium chloride, alcohol, cetyl pyridinium chloride, chloroxylenol, hexachlorophene, and chlorhexidine.

Examples of topical antifungals that may be added to the compositions of the present invention in order to control fungal growth that may be associated with the sores, include, but are not limited to, haloprogin, ciclopirox, flucytosine, miconazole, econazole, clotrimazole, fluconazole, oxiconazole, sulconazole, metronidazole, itraconazole, ketoconazole, butaconazole, terconazole, nystatin, povidone-iodine, tolnaftate, terbinafine hydrochloride, micatin, nystatin, amphorericin B, griseofulvin, benzoic acid, salicylic acid, mercuric oxide, resorcinol, triacetin, undecylenic acid and its calcium, copper and zinc salts.

### EXAMPLES

The following preparations and examples are given to enable those skilled in the art to more clearly understand and to practice the present invention. They should not be considered as limiting the scope of the invention, but merely as being illustrative and representative thereof.

### Example 1

### HSV-1 Assay

Vero cells (ATCC CCL-81) are pregrown in 96-well tissue culture plates using Dulbecco's Modified Eagle's Medium (DMEM) supplemented with 10% heat-inactivated fetal bovine serum (FBS), L-Glutamine, penicillin, and streptomycin.

To each of the replicate cell cultures is added 50 μL of the test article solution and 50 μL of virus suspension (ATCC VR-260). The multiplicity of infection used is about 0.05 plaque-forming unit (PFU) per cell. Cell controls containing medium alone, virus-infected controls containing medium and virus, drug cytotoxicity controls containing medium and each drug concentration, reagent controls containing culture medium only (no cells), and the test article colorimetric controls containing the test article and medium (no cells) are run simultaneously with the test samples. The plates are incubated at 37° C. in a humidified atmosphere containing 5% CO₂ until maximum CPE (cytopathic effect) is observed in the untreated virus control cultures (Day 5).

CPE inhibition is determined by a dye (MTS) uptake procedure (Promega's Cell Titer Aqueous One Solution). This method measures cell viability and is based on the reduction of the tetrazolium-based MTS by mitochondrial enzymes of viable host cells to MTS formazan. MTS (10 μl) is added to each of the plate wells. The plates are incubated at 37° C. for 4 hours. The purple color of the MTS formazan is then measured spectrophotometrically at 490/650 nm. The optical density (OD) value of each culture is a function of the amount of formazan produced which is proportional to the number of viable cells.

The percent of CPE (cytopathic effect) reduction of the virus-infected wells (antiviral efficacy) was measured and calculated, following which an IC₅₀ (inhibitory concentration at which the extract provides 50% CPE reduction) was then calculated.

The pomegranate (Punica granatum) extract was made from the aerial parts and obtained from PhytoMyco Research Corporation (Greenville, N.C.). The green tea (Camellia sinensis) extract was obtained from LKT Labs Inc. (St. Paul, Minn.).

Extracts of the present invention when tested as described above showed reduction of viral replication as depicted in Table 1.

TABLE 1

| | IC₅₀ |
|---|---|
| Extract | μg/mL |
| Green Tea Extract | 72.4 |
| Pomegranate extract | 86.5 |
| Grean Tea + Pomegranate (1:1) | 52.2 |

As is shown in Table 1, extracts of the present invention tested separately exhibited moderate anti-herpes replication activity. However, combining the extracts of the present invention resulted in an unexpected, greater synergistic CPE reduction.

1. A method of treating a viral lesion comprising administering to a subject in need of such treatment a composition comprising an extract of pomegranate and an extract of green tea.

2. The method of claim 1, wherein the viral lesion is caused by herpes simplex.

3. The method of claim 1, wherein the viral lesion is a cold sore.

4. The method of claim 1, wherein the composition is administered topically to the lesion.

5. The method of claim 1, wherein the composition is administered orally.

6. The method of claim 1, wherein the composition further comprises at least one agent selected from one of the following groups: (i) a skin protectant active ingredient selected from allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, and/or zinc oxide, and (ii) an external analgesic, anesthetic or antipruritic ingredient selected form benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate and/or diphenydramine hydrochloride.

7. The method of claim 1, wherein the composition further comprises at least one other agent selected from the group consisting of anti-microbial agents, other antiviral agents, antifungal agents, antioxidants, buffering agents, sunscreens, cosmetic agents, fragrances, lubricants, moisturizers, drying agents, and thickening agents.

8. A method of treating a symptom associated with viral infection in a subject in need to such treatment, comprising administering to the subject a composition comprising an extract of pomegranate and an extract of green tea.

9. The method of claim 8, wherein the viral infection is caused by herpes simplex.

10. The method of claim 8, wherein the symptom is selected from the group consisting of fever, muscle aches, swollen glands, malaise, itching, inflammation, irritation, pain, swelling and burning.

11. The method of claim 8, wherein the composition is administered topically.

12. The method of claim 8, wherein the composition is administered orally.

13. The method of claim 8, wherein the composition further comprises at least one agent selected from one of the following groups: (i) a skin protectant active ingredient selected from allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, and/or zinc oxide, and (ii) an external analgesic, anesthetic or antipruritic ingredient selected form benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate and/or diphenydramine hydrochloride.

14. The method of claim 8, wherein the composition further comprises at least one other agent selected from the group consisting of anti-microbial agents, other antiviral agents, antifungal agents, antioxidants, buffering agents, sunscreens, cosmetic agents, fragrances, lubricants, moisturizers, drying agents, and thickening agents.

15. A method for controlling viral growth and replication resulting from herpes simplex virus comprising administering to a subject in need of such treatment a composition comprising an extract of pomegranate and an extract of green tea.

16. (canceled)

17. The method of claim 15, wherein the composition is administered topically.

18. The method of claim 15, wherein the composition is administered orally.

19. The method of claim 15, wherein the composition further comprises at least one agent selected from one of the following groups: (i) a skin protectant active ingredient selected from allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, and/or zinc oxide; and (ii) an external analgesic, anesthetic or antipruritic ingredient selected form benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate, and/or diphenydramine hydrochloride.

20. The method of claim 15, wherein the composition further comprises at least one other agent selected from the group consisting of anti-microbial agents, other antiviral agents, antifungal agents, antioxidants, buffering agents, sunscreens, cosmetic agents, fragrances, lubricants, moisturizers, drying agents, and thickening agents.
